(19) United States
(12) Patent Application Publication
(10) Pub. No.: US 2021/0161701 A1
(43) Pub. Date: Jun. 3, 2021

(54) BOWEL WASTE MANAGEMENT SYSTEMS AND METHODS FOR USE

(71) Applicant: Airway Medix S.A., Warsaw (PL)

(72) Inventor: Oron ZACHAR, Tel Aviv (IL)

(21) Appl. No.: 17/092,307

(22) Filed: Nov. 8, 2020

Related U.S. Application Data
(60) Provisional application No. 62/933,425, filed on Nov. 9, 2019.

(51) Int. Cl. A61F 5/451; A61F 5/44; A61F 5/00

(52) U.S. Cl. CPC A61F 5/451; A61F 5/0093; A61F 5/4408

(57) ABSTRACT

A bowel management system comprises a waste-transport device including a collection member for bowel drainage and an inflatable retention cuff mounted around the collection member to define fixed proximal and distal cuff-attachment locations on the surface of the collection member. System characteristics include: when the mounted cuff is inflated to a pressure of 10 cm H2O in free space, a maximum diameter of the cuff is greater than 30 mm, and when the mounted cuff is inflated in free space to a pressure of at least 25 cm H2O and at most 60 cm H2O, a maximum diameter of the cuff is at least 65 mm, and/or at least 1.5 times the maximum diameter of the cuff when inflated in free space to a pressure of at most 10 cm H2O.

### FIELD OF THE INVENTION

The present invention relates to bowel waste management systems based on collection members having inflatable retention cuffs.

### SUMMARY

A method is disclosed, according to embodiments, of employing a bowel management system in the course of treatment of a human patient. The method comprises: (a) providing a waste transport device that includes (i) a collection member for bowel drainage, the collection member comprising a distal end, a proximal end and a collapsible transsphincteric zone, and (ii) an inflatable retention cuff mounted around the collection member, the cuff being distally displaced from the collapsible transsphincteric zone; and (b) deploying at least a distal portion of the collection member within the patient's rectum so that the mounted retention cuff is disposed therein and the collapsible transsphincteric zone is disposed across the patient's sphincter, wherein the mounted retention cuff is characterized by (i) having a maximum diameter greater than 30 mm when inflated in free space to a pressure of 10 cm H₂O, and (ii) having a maximum diameter, when inflated in free space to a pressure of at least 25 cm H₂O and at most 60 cm H₂O, of at least 65 mm and/or at least 1.5 times a maximum diameter of said mounted retention cuff when inflated in free space to a pressure of at most 10 cm H₂O.

In some embodiments of the method, the collection member can be sized for an adult human rectum.

In some embodiments of the method, it can be that when the cuff is inflated to a pressure of 10 cm H₂O, and is deployed within the adult human rectum so that the collection member is co-axial with the human rectum, a widest portion of the cuff is in wrinkled contact with the rectum, and the cuff is unstretched at the wrinkled contact with the rectum.

In some embodiments of the method, when the mounted cuff is uninflated or inflated to a pressure of 10 cm H₂O, and is deployed within the adult human rectum so that the collection member is co-axial with the human rectum, the diameter of the cuff at a widest portion of the cuff can be greater than 30 mm and less than 60 mm.

In some embodiments of the method, when the waste-transport device is in free space, it can be that the inflatable cuff cannot be air-inflated to a pressure of 60 cm H₂O.

In some embodiments of the method, when the waste-transport device is in free space, it can be, that the inflatable cuff is operable to be air-inflated to a pressure of 35 cm H₂O such that during a pressure ramp-up, after reaching a pressure of 20 cm H₂O an additional 4 cc or an additional 5 cc or more of air is required in order to reach a pressure of 35 cm H₂O.

In some embodiments of the method, it can be that when the mounted cuff is deployed within a rigid in-vitro enclosing tube having an inner diameter of at least 40 mm and not more than 50 mm so that (i) the collection member is co-axial with the rigid in-vitro enclosing tube and (ii) the tube-mounted cuff is uninflated or inflated to a pressure of 10 cm H₂O, a portion of the mounted cuff is in wrinkled contact with an inner wall of the rigid in-vitro enclosing tube.

In some embodiments of the method, it can be that when the mounted cuff is deployed coaxially within the in-vitro enclosing tube and inflated to a pressure of 10 cm H₂O, a ratio between (i) a length of a band of the cuff in wrinkle-free contact with the in-vitro enclosing tube and (ii) a length of a band of the cuff in wrinkled contact with the in-vitro enclosing tube, is at most 0.01 or at most 0.1 or at most 0.2.

In some embodiments of the method, it can be that when the mounted cuff is deployed coaxially within the in-vitro enclosing tube and inflated to a pressure of 40 cm H₂O, a ratio between (i) a length of the cuff in wrinkle-free contact with the in-vitro enclosing tube and (ii) a length of the cuff in in wrinkled contact with the in vitro enclosing tube is at least 0.3 or at least 0.5 or at least 1.

In some embodiments of the method, it can be that throughout a 2-cm long portion of the cuff with a longitudinal center midway between the proximal and distal cuff attachment locations, a Shore A material-hardness value of the cuff material is at most 30 or at most 20 or at most 10.

In some embodiments of the method, it can be that wherein throughout a 2-cm long portion of the cuff with a longitudinal center midway between the proximal and distal cuff attachment locations, a Shore A material-hardness value of the cuff material is at least 4 or least 5.

According to embodiments disclosed herein, a bowel management system comprises: (a) a waste-transport device including a collection member for bowel drainage, the collection member comprising (i) a distal end and a proximal end, and (ii) a collapsible transsphincteric zone, the collection member being configured such that during normal use, the distal end of the collection member is disposed within a patient's rectum and the collapsible transsphincteric zone is disposed across the patient's sphincter; and (b) an inflatable retention cuff mounted around the collection member so as to define fixed proximal and distal cuff-attachment locations on the surface of the collection member, the proximal cuff-attachment location being distally displaced from the transsphincteric zone and the distal cuff-attachment location being at, or proximally displaced from, the distal end, wherein: (i) when the mounted cuff is inflated to a pressure of 10 cm H₂O in free space, a maximum diameter of the cuff is greater than 30 mm; and (ii) when the mounted cuff is inflated in free space to a pressure of at least 25 cm H₂O and at most 60 cm H₂O, a maximum diameter of the cuff is at least 65 mm, and/or at least 1.5 times the maximum diameter of the cuff when inflated in free space to a pressure of at most 10 cm H₂O.

In some embodiments, the collection member can be sized for an adult human rectum.

In some embodiments, it can be that when the cuff is inflated to a pressure of 10 cm H₂O, and is deployed within the adult human rectum so that the collection member is co-axial with the human rectum, a widest portion of the cuff is in wrinkled contact with the rectum, and the cuff is unstretched at the wrinkled contact with the rectum.

In some embodiments, it can be that when the mounted cuff is uninflated or inflated to a pressure of 10 cm H₂O, and is deployed within the adult human rectum so that the collection member is co-axial with the human rectum, the diameter of the cuff at a widest portion of the cuff is greater than 30 mm and less than 60 mm.

In some embodiments, it can be that when the waste-transport device is in free space, the inflatable cuff cannot be air-inflated to a pressure of 60 cm H₂O.

In some embodiments, it can be that when the waste-transport device is in free space, the inflatable cuff is operable to be air-inflated to a pressure of 35 cm H₂O such that during a pressure ramp-up, after reaching a pressure of 20 cm H₂O an additional 4 cc or an additional 5 cc or more of air is required in order to reach a pressure of 35 cm H₂O.

In some embodiments, it can be that when the mounted cuff is deployed within a rigid in-vitro enclosing tube having an inner diameter of at least 40 mm and not more than 50 mm so that (i) the collection member is co-axial with the rigid in-vitro enclosing tube and (ii) the tube-mounted cuff is uninflated or inflated to a pressure of 10 cm H₂O, a portion of the mounted cuff is in wrinkled contact with an inner wall of the rigid in-vitro enclosing tube.

In some embodiments, it can be that when the mounted cuff is deployed coaxially within the in-vitro enclosing tube and inflated to a pressure of 10 cm H₂O, a ratio between (i) a length of a band of the cuff in wrinkle-free contact with the in-vitro enclosing tube and (ii) a length of a band of the cuff in wrinkled contact with the in-vitro enclosing tube, is at most 0.01 or at most 0.1 or at most 0.2.

In some embodiments, it can be that when the mounted cuff is deployed coaxially within the in-vitro enclosing tube and inflated to a pressure of 40 cm H₂O, a ratio between (i) a length of the cuff in wrinkle-free contact with the in-vitro enclosing tube and (ii) a length of the cuff in in wrinkled contact with the in vitro enclosing tube is at least 0.3 or at least 0.5 or at least 1.

In some embodiments, it can be that throughout a 2-cm long portion of the cuff with a longitudinal center midway between the proximal and distal cuff attachment locations, a Shore A material-hardness value of the cuff material is at most 30 or at most 20 or at most 10.

In some embodiments, it can be that throughout a 2-cm long portion of the cuff with a longitudinal center midway between the proximal and distal cuff attachment locations, a Shore A material-hardness value of the cuff material is at least 4 or least 5.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. Also, in some drawings the relative sizes of objects, and the relative distances between objects, may be exaggeratedly large or small for the sake of convenience and clarity of presentation. In the drawings:

FIG. 1 is a perspective view of a bowel waste management system according to embodiments of the present invention.

FIG. 2 shows a diagrammatic cross-section of portions of a human's anatomy, having portions of a bowel waste management system superimposed thereupon, according to embodiments of the present invention.

FIGS. 3-5 are schematic drawings of a retention cuff deployed on a distal portion of a waste transport device, the cuff inflated under a variety of conditions, according to embodiments of the present invention.

FIG. 6 illustrates a relationship between a contact length and a pressure parameter according to embodiments of the present invention.

FIG. 7 shows a flowchart of a method of employing a bowel management system in the course of treatment of a human patient, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

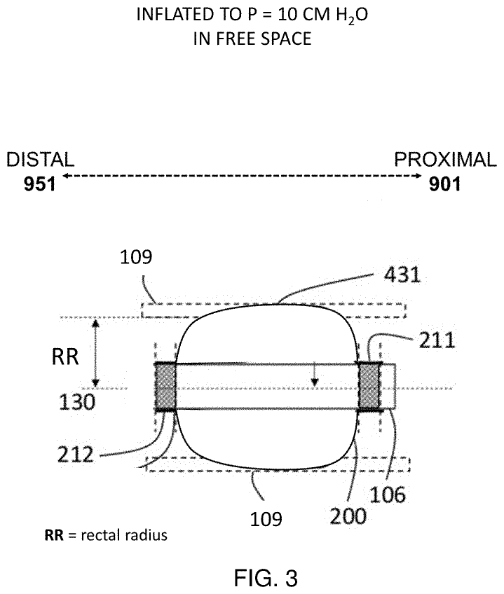

The terms ‘distal’ and ‘proximal’ are to be understood as follows: the end of a collection member intended for insertion into a human patient's rectum is the distal end of the member, and the opposite end is the proximal end. FIGS. 3-5 show distal direction 951 and proximal direction 901 with respect to a distal portion of collection member 106, both of these directions being in keeping with the preceding explanation.

There is a need for a bowel waste management system having a collection tube with an inflatable cuff that has improved pressure stability and/or good contact even at low pressure, for example to handle the fact that (a) the rectum is not a perfect cylinder and (b) there is some variance in the diameter of human rectums.

Embodiments of the present invention relate to apparatus and methods for deploying an inflatable rectal retention cuff achieving a relatively stable cuff pressure, within the range of 25-60 cm H₂O, under varying volume on the order of 10% of the cuff volume.

Unlike previously known art of bowel waste tube retention cuffs, the present invention introduces cuffs that behave significantly different when inflated within an enclosing tube compared with free space inflation. The cuffs' pressure curve is different when inflated within different tube diameters.

For the present disclosure and as shown in the figures, it is assumed that when drain tube 106 is in enclosing tube 108 (i.e. either a human rectum or an in-vitro enclosing tube), the drain tube is co-axial with the enclosing tube 108. For the present disclosure, any feature disclosed with respect to a human rectum may also be provided with respect to an external ‘in vitro’ rigid straight enclosing (i.e. perfectly cylindrical) test tube whose interior width matches that of an adult human rectum similar in size to an in vitro' rigid straight (i.e. perfectly cylindrical) test tube whose interior width/diameter is 50 mm. +/−1 mm or +/−2 mm or +/−3 mm.

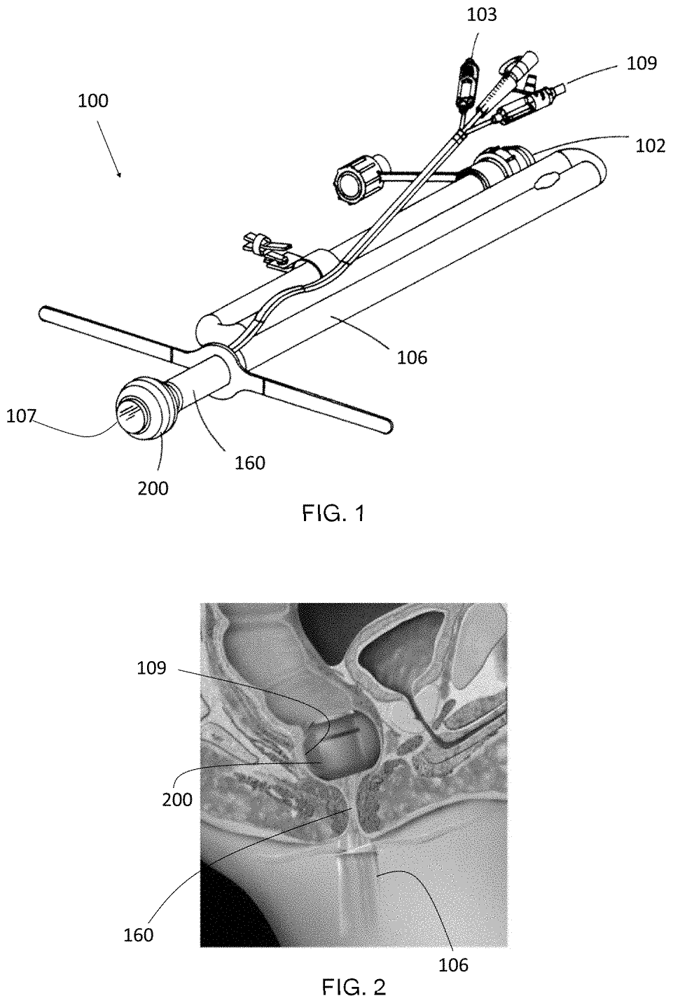

Referring now to the figures, and in particular FIG. 1, a bowel management system 100 according to embodiments comprises a waste-transport device which includes a collection member 106 for bowel drainage. The collection member comprises (i) a distal end 107 and a proximal end 102, and (ii) a collapsible transsphincteric zone 160. The collection member 106 is configured such that during normal use, the distal end 107 of the collection member 106 is disposed within a patient's rectum and the collapsible transsphincteric zone 160 is disposed across the patient's sphincter. The waste transport device additionally comprises an inflatable retention cuff 200 mounted around the collection member 106 so as to define fixed proximal and distal cuff-attachment locations 212, 211 (shown in FIG. 3) on the surface of the collection member 106. The proximal cuff-attachment location 212 is distally displaced from the transsphincteric zone 160, and the distal cuff-attachment location 211 is either at the distal end 107 or proximally displaced therefrom.

A typical cuff inflation inlet 109 is shown, and can be used for air inflation and/or liquid inflation, e.g., by using tap water or saline solution.

In some embodiments, the bowel management system 100 can also include one or more inlets 109 for one or more optional lumens.

We refer now to FIG. 2, which shows a cross-sectional diagram of a human rectum and sphincter, with details of the collection member 106 superimposed thereupon. The collection member 106 is preferably sized for an adult human rectum. The retention cuff 200 is shown as suitably inflated for contact with the rectum wall 109.

According to embodiments, an exemplary characteristic of the present invention is that when the retention cuff 200 is uninflated or inflated to a pressure of up to 10 cm H₂O, and is deployed within an adult human rectum so that the collection member 106 is co-axial with the human rectum, a widest portion 431 of the retention cuff 200 is in wrinkled contact with a rectum wall 109, and the retention cuff 200 is unstretched at the wrinkled contact with the rectum. FIG. 3 illustrates the inflation of a retention cuff 200 (mounted on collection member 106), to 10 cm H₂O in free space. A longitudinal center line 130 of the collection member is shown as a dotted line. Dashed lines show where the rectum walls 109 would be in the case of inflation within a rectum and not in free space. The measure ‘RR’ indicates the radius of a typical adult human rectum, i.e., being equal to the distance between tube centerline 130 and the dashed-line representation of the location of the rectum wall 108. According to embodiments, when the mounted retention cuff 200 is uninflated or inflated to a pressure of 10 cm H₂O, and is deployed within the adult human rectum so that the collection member 106 is co-axial with the human rectum, a maximum diameter of the cuff 200 (the diameter of the cuff 200 at a widest portion of the cuff 200) is greater than 30 mm and less than 60 mm.

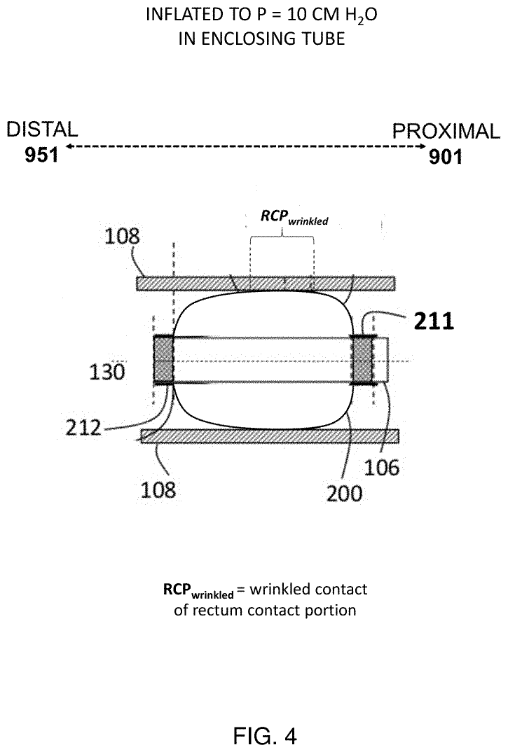

FIG. 4 shows the retention cuff 200 of FIG. 3, once again inflated to 10 cm H₂O, but this time within a solid cylindrical enclosing tube 108, preferably one having an inner diameter of 50 mm or approximately 50 mm. FIG. 4 thus illustrates the length of contact between the moderately inflated cuff 200 with the wall of the enclosing tube as RCP_{WRINKLED}, i.e., wrinkled contact with the rectum contact portion. According to some embodiments, when the mounted retention cuff 200 is deployed coaxially within the in-vitro enclosing tube 108 and inflated to a pressure of 10 cm H₂O, a ratio between (i) a length of a band of the cuff 200 in wrinkle-free contact with the in-vitro enclosing tube 108 and (ii) a length of a band of the cuff in wrinkled contact with the in-vitro enclosing tube 108, is at most 0.01 or at most 0.1 or at most 0.2. According to some embodiments, when the mounted retention cuff 200 is deployed coaxially within the in-vitro enclosing tube 108 and inflated to a pressure of 40 cm H₂O, a ratio between (i) a length of the cuff 200 in wrinkle-free contact with the in-vitro enclosing tube 108 and (ii) a length of the cuff 200 in wrinkled contact with the in vitro enclosing tube 108 is at least 0.3 or at least 0.5 or at least 1.

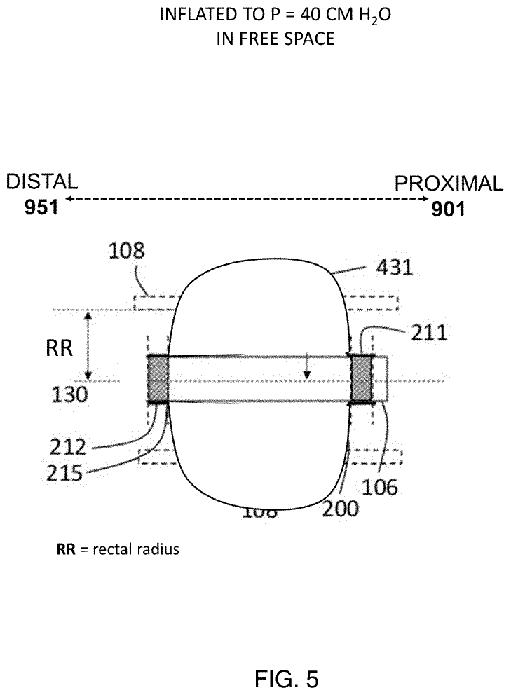

In some embodiments, when the mounted retention cuff 200 is inflated in free space to a pressure of at least 25 cm H₂O and at most 60 cm H₂O, a maximum diameter of the cuff 200 is at least 65 mm. In some embodiments, a maximum diameter of the cuff 200 is at least 1.5 times the maximum diameter of the cuff 200 when inflated in free space to a pressure of at most 10 cm H₂O (shown in FIG. 3). FIG. 5 illustrates the retention cuff 200 of FIGS. 3 and 4 inflated to a pressure of 40 cm H₂O in free space.

In embodiments, the inflatable retention cuff 200 is characterized by not being able to be air-inflated to a pressure of 60 cm H₂O when the collection member 106 of the waste-transport device is in free space (or the waste-transport device is in free space).

In some embodiments, the inflatable retention cuff 200 is characterized by being operable to be air-inflated to a pressure of 35 cm H₂O such that during a pressure ramp-up, after reaching a pressure of 20 cm H₂O, an additional 4 cc or an additional 5 cc or more of air is required in order to reach a pressure of 35 cm H₂O.

In embodiments, the inflatable retention cuff 200 is characterized by a material hardness such that throughout a 2-cm long portion of the cuff 200 with a longitudinal center 130 midway between the proximal and distal cuff attachment locations 211, 212, a Shore A material-hardness value of the cuff material is at most 30 or at most 20 or at most 10.

In embodiments, the inflatable retention cuff 200 is characterized by a material hardness such that throughout a 2-cm long portion of the cuff 200 with a longitudinal center 130 midway between the proximal and distal cuff attachment locations 211, 212, a Shore A material-hardness value of the cuff material is at least 4 or least 5.

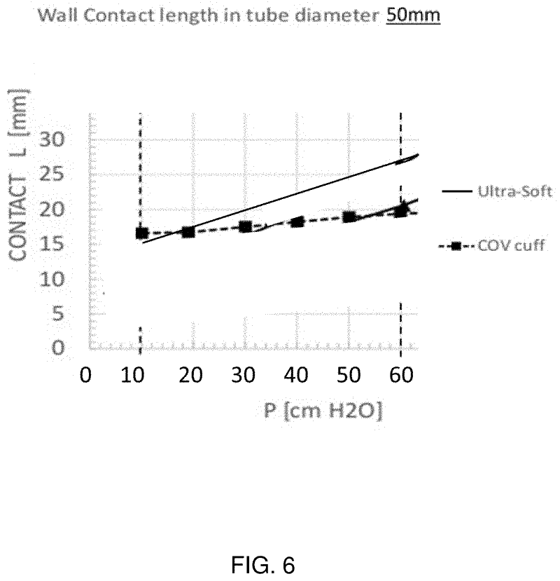

According to embodiments of the invention, contact length between an inflated cuff 200 (e.g., inflated with air or with a liquid such as tap water or a saline solution) and the wall of an enclosed 50 mm tube 108 increases substantially linearly with pressure, i.e., when the cuff 200 is inflated while deployed in the enclosing tube 108. FIG. 6 shows a graph comparing the contact length-pressure curve of cuffs 200 according to the present invention (indicated by ‘Ultra-Soft’ in FIG. 6) and a prior art cuff arrangement (indicated by ‘COV cuff’ in FIG. 6). As shown by the graph, the increase in contact length vs. pressure is substantially greater in the case of the ‘Ultra-Soft’ cuffs, i.e., the cuffs of the present invention.

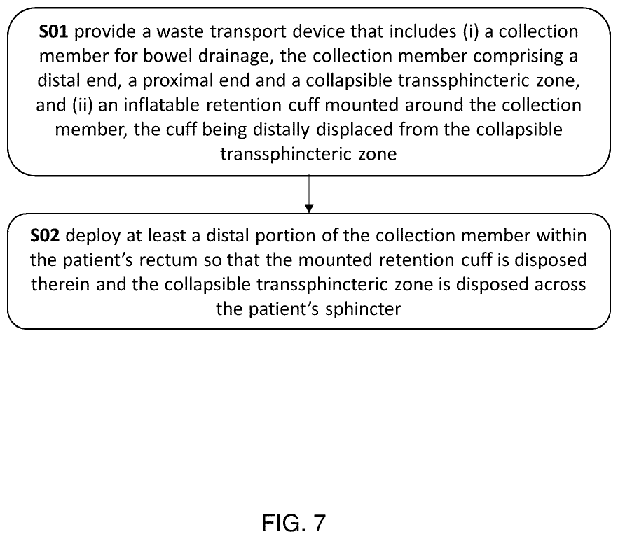

A method is now disclosed for employing a bowel management system in the course of treatment of a human patient. As illustrated in the flowchart of FIG. 7, The method comprises:

Step S01 providing a waste transport device that includes (i) a collection member 106 for bowel drainage, the collection member 106 comprising a distal end 107, a proximal end 109 and a collapsible transsphincteric zone 160, and (ii) an inflatable retention cuff 200 mounted around the collection member 106, the cuff 200 being distally displaced from the collapsible transsphincteric zone 160; and

Step S02 deploying at least a distal portion of the collection member 106 within the patient's rectum so that the mounted retention cuff 200 is disposed therein and the collapsible transsphincteric zone 160 is disposed across the patient's sphincter.

According to the method, the mounted retention cuff 200 is characterized by (i) having a maximum diameter greater than 30 mm when inflated in free space to a pressure of 10 cm H₂O, and (ii) having a maximum diameter, when inflated in free space to a pressure of at least 25 cm H₂O and at most 60 cm H₂O, of at least 65 mm and/or at least 1.5 times a maximum diameter of said mounted retention cuff 200 when inflated in free space to a pressure of at most 10 cm H₂O.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. A method of employing a bowel management system in the course of treatment of a human patient, the method comprising:
a. providing a waste transport device that includes (i) a collection member for bowel drainage, the collection member comprising a distal end, a proximal end and a collapsible transsphincteric zone, and (ii) an inflatable retention cuff mounted around the collection member, the cuff being distally displaced from the collapsible transsphincteric zone; and
b. deploying at least a distal portion of the collection member within the patient's rectum so that the mounted retention cuff is disposed therein and the collapsible transsphincteric zone is disposed across the patient's sphincter,
wherein the mounted retention cuff is characterized by (i) having a maximum diameter greater than 30 mm when inflated in free space to a pressure of 10 cm H₂O, and (ii) having a maximum diameter, when inflated in free space to a pressure of at least 25 cm H₂O and at most 60 cm H₂O, of at least 65 mm and/or at least 1.5 times a maximum diameter of said mounted retention cuff when inflated in free space to a pressure of at most 10 cm H₂O.

2. The method of claim 1 wherein the collection member is sized for an adult human rectum.

3. The method of claim 2, wherein when the cuff is inflated to a pressure of 10 cm H₂O, and is deployed within the adult human rectum so that the collection member is co-axial with the human rectum, a widest portion of the cuff is in wrinkled contact with the rectum, and the cuff is unstretched at the wrinkled contact with the rectum.

4. The method of claim 2, wherein when the mounted cuff is uninflated or inflated to a pressure of 10 cm H₂O, and is deployed within the adult human rectum so that the collection member is co-axial with the human rectum, the diameter of the cuff at a widest portion of the cuff is greater than 30 mm and less than 60 mm.

5. The method of claim 1, wherein when the waste-transport device is in free space, the inflatable cuff cannot be air-inflated to a pressure of 60 cm H₂O.

6. The method of claim 1, wherein when the waste-transport device is in free space, the inflatable cuff is operable to be air-inflated to a pressure of 35 cm H₂O such that during a pressure ramp-up, after reaching a pressure of 20 cm H₂O an additional at least 4 cc air is required in order to reach a pressure of 35 cm H₂O.

7. The method of claim 1, wherein when the mounted cuff is deployed within a rigid in-vitro enclosing tube having an inner diameter of at least 40 mm and not more than 50 mm so that (i) the collection member is co-axial with the rigid in-vitro enclosing tube and (ii) the tube-mounted cuff is uninflated or inflated to a pressure of 10 cm H₂O, a portion of the mounted cuff is in wrinkled contact with an inner wall of the rigid in-vitro enclosing tube.

8. The method of claim 7, wherein when the mounted cuff is deployed coaxially within the in-vitro enclosing tube and inflated to a pressure of 10 cm H₂O, a ratio between (i) a length of a band of the cuff in wrinkle-free contact with the in-vitro enclosing tube and (ii) a length of a band of the cuff in wrinkled contact with the in-vitro enclosing tube, is at most 0.2.

9. The method of claim 7, wherein when the mounted cuff is deployed coaxially within the in-vitro enclosing tube and inflated to a pressure of 40 cm H₂O, a ratio between (i) a length of the cuff in wrinkle-free contact with the in-vitro enclosing tube and (ii) a length of the cuff in in wrinkled contact with the in vitro enclosing tube is at least 0.3.

10. The method of claim 1, wherein throughout a 2-cm long portion of the cuff with a longitudinal center midway between the proximal and distal cuff attachment locations, a Shore A material-hardness value of the cuff material is at most 30 and/or at least 4.

11. A bowel management system comprising:
a. a waste-transport device including a collection member for bowel drainage, the collection member comprising (i) a distal end and a proximal end, and (ii) a collapsible transsphincteric zone, the collection member being configured such that during normal use, the distal end of the collection member is disposed within a patient's rectum and the collapsible transsphincteric zone is disposed across the patient's sphincter; and
b. an inflatable retention cuff mounted around the collection member so as to define fixed proximal and distal cuff-attachment locations on the surface of the collection member, the proximal cuff-attachment location being distally displaced from the transsphincteric zone and the distal cuff-attachment location being at, or proximally displaced from, the distal end, wherein:
(i) when the mounted cuff is inflated to a pressure of 10 cm H₂O in free space, a maximum diameter of the cuff is greater than 30 mm; and
(ii) when the mounted cuff is inflated in free space to a pressure of at least 25 cm H₂O and at most 60 cm H₂O, a maximum diameter of the cuff is at least 65 mm, and/or at least 1.5 times the maximum diameter of the cuff when inflated in free space to a pressure of at most 10 cm H₂O.

12. The bowel management system of claim 11, wherein the collection member is sized for an adult human rectum.

13. The bowel management system of claim 12, wherein when the cuff is inflated to a pressure of 10 cm H₂O, and is deployed within the adult human rectum so that the collection member is co-axial with the human rectum, a widest portion of the cuff is in wrinkled contact with the rectum, and the cuff is unstretched at the wrinkled contact with the rectum.

14. The bowel management system of claim 12, wherein when the mounted cuff is uninflated or inflated to a pressure of 10 cm H₂O, and is deployed within the adult human rectum so that the collection member is co-axial with the human rectum, the diameter of the cuff at a widest portion of the cuff is greater than 30 mm and less than 60 mm.

15. The bowel management system of claim 11, wherein when the waste-transport device is in free space, the inflatable cuff cannot be air-inflated to a pressure of 60 cm H₂O.

16. The bowel management system of claim 11, wherein when the waste-transport device is in free space, the inflatable cuff is operable to be air-inflated to a pressure of 35 cm H₂O such that during a pressure ramp-up, after reaching a pressure of 20 cm H₂O an additional at least 4 cc of air is required in order to reach a pressure of 35 cm H₂O.

17. The bowel management system of claim 11, wherein when the mounted cuff is deployed within a rigid in-vitro enclosing tube having an inner diameter of at least 40 mm and not more than 50 mm so that (i) the collection member is co-axial with the rigid in-vitro enclosing tube and (ii) the tube-mounted cuff is uninflated or inflated to a pressure of 10 cm H₂O, a portion of the mounted cuff is in wrinkled contact with an inner wall of the rigid in-vitro enclosing tube.

18. The bowel management system of claim 17 wherein when the mounted cuff is deployed coaxially within the in-vitro enclosing tube and inflated to a pressure of 10 cm H₂O, a ratio between (i) a length of a band of the cuff in wrinkle-free contact with the in-vitro enclosing tube and (ii) a length of a band of the cuff in wrinkled contact with the in-vitro enclosing tube, is at most 0.2.

19. The bowel management system of claim 17, wherein when the mounted cuff is deployed coaxially within the in-vitro enclosing tube and inflated to a pressure of 40 cm H₂O, a ratio between (i) a length of the cuff in wrinkle-free contact with the in-vitro enclosing tube and (ii) a length of the cuff in in wrinkled contact with the in vitro enclosing tube is at least 0.3.

20. The bowel management system of claim 11, wherein throughout a 2-cm long portion of the cuff with a longitudinal center midway between the proximal and distal cuff attachment locations, a Shore A material-hardness value of the cuff material is at most 30 and/or at least 4.
